(19) United States
(12) Patent Application Publication
(10) Pub. No.: US 2025/0262396 A1
(43) Pub. Date: Aug. 21, 2025

(54) PRESSURE SUPPORT SYSTEM INCLUDING A DUAL MATERIAL DUCT STRUCTURE FOR SOUND ATTENUATION

(71) Applicant: KONINKLIJKE PHILIPS N.V., EINDHOVEN (NL)

(72) Inventor: CHRISTOPHER RANDALL BAKER, HUNTINGDON, MA (US)

(21) Appl. No.: 19/053,567

(22) Filed: Feb. 14, 2025

Related U.S. Application Data
(60) Provisional application No. 63/554,249, filed on Feb. 16, 2024.

(51) Int. Cl. A61M 16/00

(52) U.S. Cl. CPC A61M 16/00; A61M 2202/0007; A61M 2202/02; A61M 2205/0216; A61M 2205/42

(57) ABSTRACT

A pressure support system includes a housing including a base duct structure defining a flow path for a flow of breathing gas, and a dual material duct structure non-fixedly coupled to the base duct structure, wherein the dual material duct structure includes a rigid portion made of a rigid material and having a cavity defined therein, and a compliant portion made of a compliant material, wherein the compliant portion is coupled to the rigid portion and contacts the base duct structure.

### FIELD OF THE INVENTION

The disclosed concept relates generally to pressure support systems for delivering a flow of a breathing gas to the airways of a patient, and, in particular, to a dual material duct structure that may be used in the housing of a precursor support system or another system that delivers pressurized gas for attenuation of airborne sound resulting from the flow of the gas through the housing.

### BACKGROUND OF THE INVENTION

Today, the first line therapy for patients diagnosed with obstructive sleep apnea syndrome (OSAS) is pressure assisted ventilation support, also known as PAP support or therapy, most often by continuous positive airway pressure (CPAP) therapy. Such pressure assisted ventilation support involves the placement of a respiratory patient interface device, including a mask component, on the face of a patient. The mask component may be, for example and without limitation, a nasal mask that covers the patient's nose, a nasal cushion having nasal prongs that are received within the patient's nares, a pillow-style nasal cushion that engages the patient's nares without being inserted therein, a nasal/oral mask that covers the patient's nose and mouth, or a full-face mask that covers the patient's face. The respiratory patient interface device interfaces a pressure/flow generating device (also known as a PAP device) with the airway of the patient so that a flow of breathing gas can be delivered from the pressure/flow generating device to the airway of the patient.

CPAP and other pressure assisted ventilation support systems are used in a variety of environments from hospital intensive care units to a patient's nightstand inside of their home. This environmental variation poses a challenge to the designers of such products due to the variety of temperature, humidity, and atmospheric conditions that exist. There is also a desire to be able to readily clean the patient airpath. Furthermore, patients, caregivers, and bed partners all experience the sound of these devices from their own unique perceptive. Their unique perspectives and expectations require precise control of sound and vibration of these devices.

One of the most common techniques to reduce airborne sound is to use sound attenuating foam. This foam converts airborne sound to heat due to friction. Most sound attenuating foams are porous and lightweight in nature to optimize acoustic performance. Unfortunately, these same material characteristics allow for the material to degrade faster than other components inside the device, which poses a risk to the patient. Also, the porous nature of the foam makes cleaning a challenge due to the complicated path that any cleaning/rinsing agents would need to navigate to ensure that all surfaces are contacted.

### SUMMARY OF THE INVENTION

These needs, and others, are met by a pressure support system that includes a housing including a base duct structure defining a flow path for a flow of breathing gas, and a dual material duct structure non-fixedly coupled to the base duct structure, wherein the dual material duct structure includes a rigid portion made of a rigid material and having a cavity defined therein, and a compliant portion made of a compliant material, wherein the compliant portion is coupled to the rigid portion and contacts the base duct structure.

In another embodiment, a method of attenuating airborne sound in a pressure support system is provided, wherein the pressure support system has a housing having a base duct structure defining a flow path for a flow of breathing gas. The method includes providing a dual material duct structure, wherein the dual material duct structure includes a rigid portion made of a rigid material and having a cavity defined therein, and a compliant portion made of a compliant material, wherein the compliant portion is coupled to the rigid portion, and non-fixedly coupling the dual material duct structure to the base duct structure in a manner wherein the compliant portion contacts the base duct structure.

### BRIEF DESCRIPTION OF THE DRAWINGS

A full understanding of the invention can be gained from the following description of the preferred embodiments when read in conjunction with the accompanying drawings in which:

FIG. 1 is a schematic diagram of a pressure support system in which the disclosed concept may be implemented according to a non-limiting exemplary embodiment;

FIG. 2 is a schematic diagram of a portion of a housing of a pressure generating device that may form part of the pressure support system of FIG. 1 according to a non-limiting exemplary embodiment of the disclosed concept;

FIGS. 3A-3D are isometric and front elevational views, respectively, of exemplary dual material duct structures according to a non-limiting exemplary embodiment of the disclosed concept; and

FIG. 4 is a cross-sectional view of the housing and duct of FIG. 2 in which an exemplary dual material duct structure of the disclosed concept is implemented.

### DETAILED DESCRIPTION OF THE INVENTION

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body.

As used herein, the term “number” shall mean one or an integer greater than one (i.e., a plurality).

As used herein, the term “rigid material” shall mean a material having a tensile

modulus of elasticity (E) that is greater than or equal to 0.5 GPa. In one particular exemplary embodiment, the rigid materials used in the disclosed concept have a tensile modulus of elasticity (E) that is greater than or equal to 0.8 GPa.

As used herein, the term “compliant material” shall mean a material having a tensile modulus of elasticity (E) that is less than 0.5 GPA, including, but not limited to viscoelastic materials. In one particular exemplary embodiment, the complaint materials used in the disclosed concept have a tensile modulus of elasticity (E) that is less than or equal to 0.1 GPa.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As described herein, the disclosed concept uses a dual material structure to form part of a duct of a device that delivers a flow of gas, such as a duct forming a part of the housing of a pressure support system. The dual material structure is comprised of a compliant material and rigid material that is coupled to the compliant material. The dual material structure is pressed into a base duct structure that defines the flow path of the duct and is retained therein by the compliant material, including select features formed as part of the compliant material. The dual material structure is not rigidly or fixedly attached to the base duct structure. In operation, as airborne sound travels down the duct, it contacts the rigid material and causes it to vibrate. This vibration is then transmitted to the compliant material which, in the exemplary embodiment, is viscoelastic in nature. The vibration energy is converted to heat by means of deformation of the compliant material and friction between the compliant material and to the base duct structure. As a result, the airborne sound is attenuated.

The dual material duct structure of the disclosed concept provides an improvement in attenuation of airborne sound that is generated during normal CPAP and ventilator use. It includes a more durable airborne sound solution, as compared to acoustic foams, that reduces the risk of patient harm from degradation. Also, it includes no porous surfaces, which will allow for easier cleaning of the airpath unlike current foam solutions. The dual material duct structure may be located on either the inlet or outlet side of a pressure support systems, such as a CPAP or ventilator.

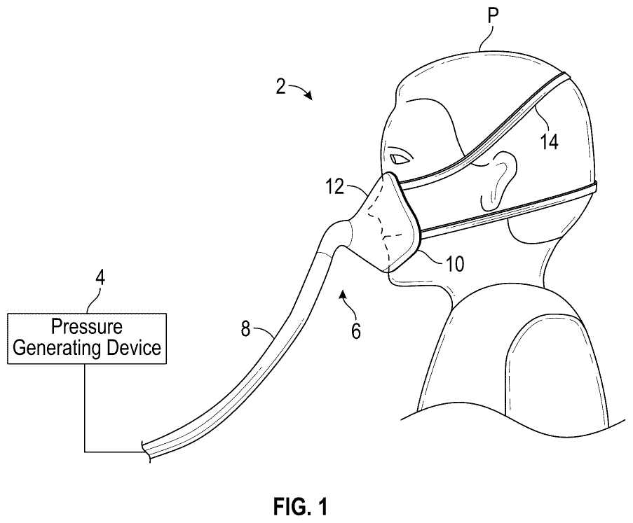

FIG. 1 is a schematic diagram of a pressure support system 2 in which the disclosed concept may be implemented according to a non-limiting exemplary embodiment. Pressure support system 2 is adapted to provide a regimen of respiratory therapy to a patient P. Pressure support system 2 includes a pressure generating device 4 (also known as a PAP device) and a patient circuit 6 including a delivery conduit 8 and a patient interface device 10. In the illustrated embodiment, patient interface device 10 includes a mask 12 and a headgear 14 for securing mask 12 to the head of patient P. In the exemplary embodiment illustrated in FIG. 1, mask 12 is a nasal/oral mask structured to be placed over the nose and mouth of patient P. Any type of mask, however, which facilitates the delivery of the flow of breathing gas to, and the removal of a flow of exhalation gas from, the airway of such a patient may be used while remaining within the scope of the disclosed concept. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Koninklijke Philips N.V.), and auto-titration pressure support devices.

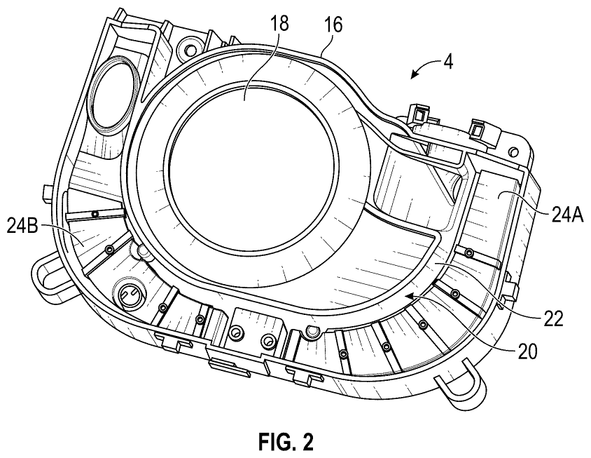

FIG. 2 is a schematic diagram of a portion of a housing 16 of pressure generating device 4 according to a non-limiting exemplary embodiment of the disclosed concept. Housing 16 includes a portion 18 structured for holding motorized blower that generates a pressured flow of breathing gas from ambient air, and a duct 20 for delivering the flow of breathing gas from housing 16 to delivery conduit 8. As described herein, duct 20 includes a base duct structure 22 that is formed as an integral part of housing 16 (that portion of housing 16 is thus a unitary structure in the exemplary embodiment), and a number of dual material duct structures 24 coupled to the base duct structure 22. In the illustrated embodiment, duct 20 includes two such dual material duct structures 24, labelled 24A and 24B.

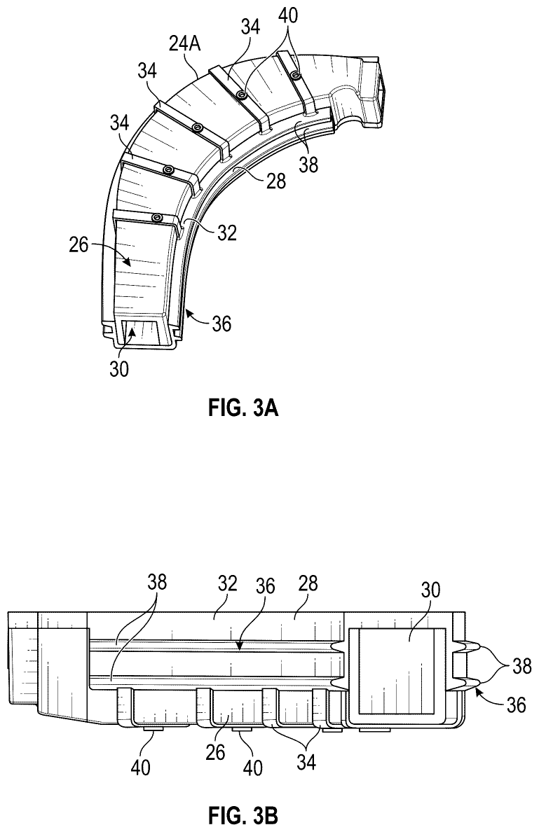

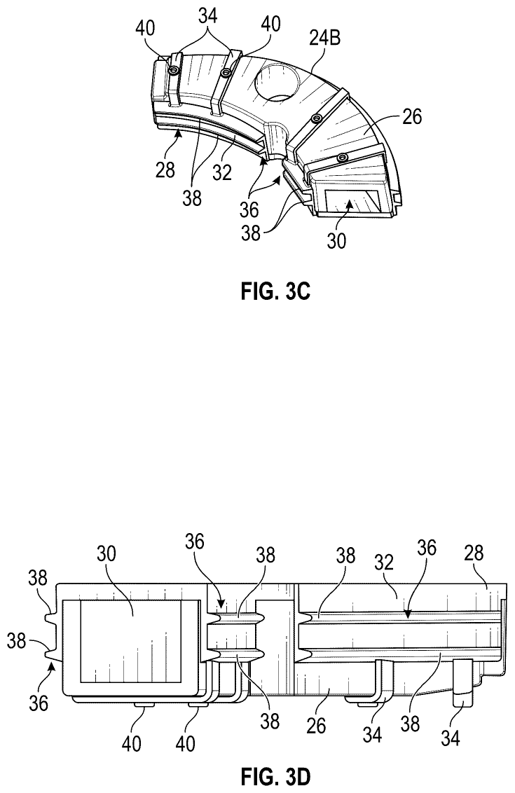

FIG. 3A is an isometric view and FIG. 3B is a front elevational view of dual material duct structure 24A according to the exemplary embodiment. FIG. 3C is an isometric view and FIG. 3D is a front elevational view of dual material duct structure 24B according to the exemplary embodiment. As seen in FIGS. 3A-3D, each dual material duct structure 24 includes a rigid portion 26 made of a rigid material and a compliant portion 28 made of a compliant material that is directly coupled to rigid portion 26. In the exemplary embodiment, dual material duct structure 24 may be formed using an over-molding processing to join the rigid material and the complaint material together. Alternatively, the rigid material and the complaint material may be joined together by an adhesive. The rigid material may, for example and without limitation, be a plastic material such as polycarbonate (GPa of about 2.6), polyethylene terephthalate (PET) (GPa of about 2.7), or polyethylene or HDPE (GPa of about 0.8). The compliant material may, for example and without limitation, be silicone rubber (GPa of about 0.000517-0.0621) or an elastic foam (GPa of about 0.01 to about 0.1).

In addition, rigid portion 26 includes a cavity 30 for allowing gas to flow through and along rigid portion 26. In the exemplary embodiment, rigid portion 26 has a U-shaped cross-section to form cavity 30. Compliant portion 28 includes a base 32 and a plurality of ribs 34 coupled to and extending from base 32. In the illustrated embodiment, base 32 has a U-Shaped cross-section for receiving rigid portion. In addition, base 32 includes a number of wiper features 36 each comprising first and second horizontal appendages 38. In the exemplary embodiment, wiper features 36 are provided on each side of base 32 and extend along the length of base 32. Also, each rib 34 includes an extension member 40 that extends outwardly therefrom. In one exemplary embodiment, each extension member 40 has a toroidal shape. In another exemplary embodiment, each extension member 40 has a cylindrical cross-section.

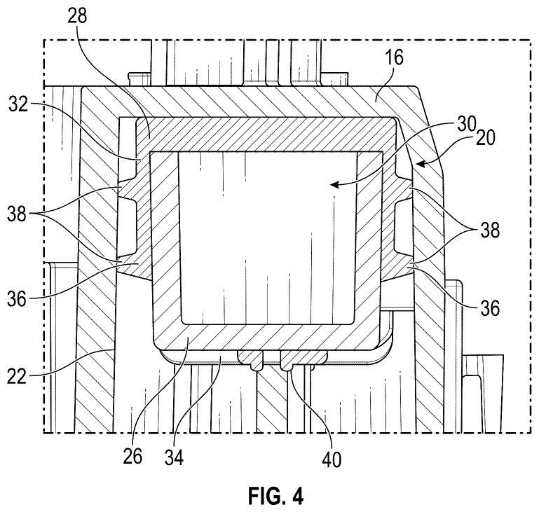

Dual material duct structure 24 is press fit into base duct structure 22. This is illustrated in FIG. 4, which is a cross-sectional view of housing 16 and duct 20. Dual material duct structure 24 is retained within base duct structure 22 by compliant portion 28, including wiper features 36, which engage the side walls of base duct structure 22, and extension members 40, which engage the other walls of base duct structure 22. Dual material duct structure 24 is not rigidly or fixedly attached to base duct structure 22. In operation, as airborne sound travels down the duct 20, it contacts rigid portion 26 and causes it to vibrate. This vibration is then transmitted to compliant potion 28. The vibration energy is converted to heat by means of deformation of the compliant material of compliant portion 28 and friction between the compliant portion 28 and base duct structure 22. Duct 20 including dual material duct structure 24 thus provides significant attenuation of the airborne sound in pressure support system 2.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. A pressure support system for delivering a flow of breathing gas to a patient, comprising:
a housing including a base duct structure defining a flow path for the flow of breathing gas; and
a dual material duct structure non-fixedly coupled to the base duct structure, wherein the dual material duct structure includes a rigid portion made of a rigid material and having a cavity defined therein, and a compliant portion made of a compliant material, wherein the compliant portion is coupled to the rigid portion and contacts the base duct structure.

2. The pressure support system according to claim 1, wherein the base duct structure and the housing comprise a unitary structure.

3. The pressure support system according to claim 1, wherein the rigid portion has a U-shaped cross-section that forms the cavity.

4. The pressure support system according to claim 1, wherein complaint portion includes a base having a U-shaped cross-section.

5. The pressure support system according to claim 4, wherein complaint portion includes a plurality of ribs coupled to and extending from the base.

6. The pressure support system according to claim 5, wherein each of the ribs includes an extension member that extends outwardly therefrom.

7. The pressure support system according to claim 4, wherein the rigid portion has a U-shaped cross-section that forms the cavity, wherein an open top end of the rigid portion contacts the base such that the rigid portion and the complaint potion together form a closed flow path for the flow of breathing gas.

8. The pressure support system according to claim 4, wherein the base has a number of wiper features each comprising a number of horizontal appendages.

9. The pressure support system according to claim 10, wherein each of the wiper features has a plurality of horizontal appendages.

10. A method of attenuating airborne sound in a pressure support system having a housing having a base duct structure defining a flow path for a flow of breathing gas, comprising:
providing a dual material duct structure, wherein the dual material duct structure includes a rigid portion made of a rigid material and having a cavity defined therein, and a compliant portion made of a compliant material, wherein the compliant portion is coupled to the rigid portion; and
non-fixedly coupling the dual material duct structure to the base duct structure in a manner wherein the compliant portion contacts the base duct structure.

11. The method according to claim 10, wherein the base duct structure and the housing comprise a unitary structure.

12. The method according to claim 10, wherein the rigid portion has a U-shaped cross-section that forms the cavity.

13. The method according to claim 10, wherein complaint portion includes a base having a U-shaped cross-section.

14. The method according to claim 10, wherein complaint portion includes a plurality of ribs coupled to and extending from the base.

15. The method according to claim 14, wherein each of the ribs includes an extension member that extends outwardly therefrom.

16. The method according to claim 10, wherein the rigid portion has a U-shaped cross-section that forms the cavity, wherein an open top end of the rigid portion contacts the base such that the rigid portion and the complaint potion together form a closed flow path for the flow of breathing gas.

17. The method according to claim 10, wherein the base has a number of wiper features each comprising a number of horizontal appendages.

18. The method according to claim 17, wherein each of the wiper features has a plurality of horizontal appendages.

19. The method according to claim 10, wherein the providing comprising forming the dual material duct structure by forming the rigid portion and the complaint portion using an over-molding process.

20. The method according to claim 10, wherein the providing comprising forming the dual material duct structure by attaching the rigid potion to the compliant portion with an adhesive.
